(19) United States
(12) Patent Application Publication
(10) Pub. No.: US 2012/0034665 A1
(43) Pub. Date: Feb. 9, 2012

(54) ENZYMATIC METHOD FOR PRODUCING ALDEHYDES

(75) Inventors: Thomas Haas, Muenster (DE); Florian Klasovsky, Haltern am See (DE); Hendrik Krauter, Nieder-Olm (DE); Steffen Schaffer, Herten (DE); Rene Schoebel, Wickede/Ruhr (DE); Thomas Tacke, Alzenau (DE); Klaus-Dieter Vorlop, Braunschweig (DE); Thomas Willke, Wendenburg-Bortfeld (DE); Mirja Wessel, Bochum (DE)

(73) Assignee: EVONIK DEGUSSA GmbH, Essen (DE)

(21) Appl. No.: 13/263,761

(22) PCT Filed: Apr. 28, 2010
(86) PCT No.: PCT/EP2010/055683

§ 371 Date: Oct. 10, 2011

(30) Foreign Application Priority Data
May 5, 2009 (DE) 10 2009 002 811.0

(51) Int. Cl. C12P 7/24; C07C 45/00

(52) U.S. Cl. 435/147; 568/449

(57) ABSTRACT

The invention relates to methods for producing aldehydes and the oxidation and reduction products thereof.

### FIELD OF THE INVENTION

The invention relates to methods for producing aldehydes and the oxidation and reduction products thereof.

### PRIOR ART

Enzymatic methods for producing aldehydes are described widely in the literature.

Thus, for example, WO 2009001304 describes a method for producing aldehydes from polyunsaturated fatty acids with cooperation of 13-hydroperoxide-lyases.

Biochemical routes for obtaining 3-methylbutanal are described in Smit et al., Appl. Microbiol. Biotechnol., 2009, 81, 987-999. Producing aldehydes from alcohols with the help of methanol oxidases and catalases from Pichia, Hansenula, Candida, or Torulopsis is described in U.S. Pat. No. 5,783,429.

Using xylene or alkane monooxygenases it is possible, according to WO 2001031047, to obtain aromatic aldehyde derivatives.

3-Hydroxypropionaldehyde (3HPA) can be converted to acrylic acid, an important monomer for the industrial production of polymers and plastics, and also to equally important acrylic acid esters. Further important products which can be derived from 3-hydroxypropionaldehyde are 1,3-propanediol (by reduction), 3-hydroxypropionic acid (by oxidation) and acrolein (by dehydration). Since to date there are no commercial sources of 3-hydroxypropionaldehyde available, the production of 3-hydroxypropionaldehyde, in particular in the form of the reuterin, from glycerol is being intensively investigated.

Thus, as early as the end of the 1980s, Talarico and Dobrogosz, Antimicrob. Agents Chemother., 1989, 33, 674-679, described the fermentative production of 3-hydroxypropionaldehyde from glycerol using Lactobacillus reuteri.

An analogous method using O₂ limitation is described in U.S. Pat. No. 5,413,960.

DK180099 discloses a method with the continuous introduction of glycerol and use of Lactobacillus reuteri DSM12246.

Doleyres et al., Appl Microbiol Biotechnol. 2005 September; 68 (4):467-74, describe a method for producing 3-hydroxypropionaldehyde in L. reuteri ATCC 53608 under various growth conditions and show that repeated use of the biocatalyst is not possible on account of the heavily decreasing activity.

CN1778935 describes a one-step fermentation of a glycerol solution with Klebsiella pneumoniae DSM2026 in the presence of semicarbazide.

U.S. Pat. No. 4,962,027 describes a method for producing 3-hydroxypropionaldehyde in an aerobic two-step fermentation of Klebsiella pneumoniae NRRL B-4011, where, in the first step, firstly cell mass and glycerol dehydratase is generated and, in the second step, in the presence of semicarbazide, glycerol is converted to 3-hydroxypropionaldehyde.

EP 1669457 describes the biotechnological production of 3-hydroxypropionaldehyde with (inter alia) K. pneumoniae with the addition of coenzyme B₁₂ in a virtually quantitative yield by increasing the cell/substrate ratio.

A common feature of all of the described methods is that the yields and conversion rates are low. Moreover, in the described methods, a high expenditure for the provision of the biocatalyst is always required, which then only has a short service life, i.e. which can then only be used in the short-term.

It was therefore an object of the invention to provide a method which overcomes the described disadvantages of the prior art.

### DESCRIPTION OF THE INVENTION

Surprisingly, it has been found that the methods described below are able to achieve the object of the invention.

The present invention therefore provides a method for producing aldehydes, comprising the steps A) bringing preferably an aqueous solution of a vicinal diol into contact with a hydro-lyase, B) separating the aldehyde formed in step A) and the hydro-lyase, C) at least one repetition of steps A) and B) with the hydro-lyase obtained from step B) and optionally D) isolating the aldehyde formed, characterized in that the method is carried out in the presence of a compound which forms a bond with the aldehyde.

The invention further provides a method for producing oxidation and reduction products of the aldehyde obtained by the aforementioned method.

Advantages of the method according to the invention are the virtually quantitative yield of aldehyde based on the alcohol used, the long service life of the hydro-lyase used as biocatalyst and its ability to be reused. This facilitates a continuous procedure.

A further advantage of the method according to the invention is that there is no need to suppress secondary activities of the hydro-lyase used as biocatalyst, such as, for example, further reduction of the aldehyde to alcohol, to the formed aldehyde.

A further advantage of the present invention is that the method according to the invention offers the option of working for the most part with the exclusion or reduction of oxygen, as a result of which corrosion and undesired oxidation for example of the product can be reduced, a simple set-up in terms of apparatus is made possible and energy can be saved, and also other known problems associated with the gassing of a biotechnological reactor such as, for example, foam formation, are prevented.

In connection with the present invention, the term “aldehyde” is likewise understood as meaning hydrates and dimers of the aldehyde and also mixtures of these compounds, and, for example, reuterin in the case of 3-hydroxypropionaldehyde.

Unless stated otherwise, all stated percentages (%) are percentages by mass.

Hydro-lyases which can be used in the method according to the invention are enzymes which are listed in the EC Class 4.2.1.X. Here, hydro-lyases which are particularly advantageous for the method according to the invention are those which are active independently of coenzyme B₁₂, i.e. which have a hydro-lyase activity even without the presence of coenzyme B₁₂ or substitutes/analogs for this substance such as, for example, corrinoids, cobalamines. Examples of representatives of such hydro-lyases can be found in WO 200814864.

The hydro-lyases preferably used in the method according to the invention are glycerol dehydratases and diol-dehydratases and also propanediol dehydratases (EC 4.2.1.30, EC 4.2.1.28). In this connection, in particular the glycerol dehydratases which can be isolated from microorganisms selected from the group of the genera Klebsiella, Citrobacter, Clostridium, Lactobacillus, Enterobacter, Caloramator, Salmonella and Listeria, particularly preferably the glycerol dehydratase selected from the group of the species Klebsiella pneumoniae, Citrobacter pneumoniae, Clostridium pasteurianum, Lactobacillus leichmannii, Citrobacter intermedium, Lactobacillus reuteri, Lactobacillus buchneri, Lactobacillus brevis, Enterobacter agglomerans, Clostridium pasteurianum, Clostridium perfringens, Clostridium kluyveri, Caloramator viterbensis, Lactobacillus collinoides, Lactobacillus hilgardii, Salmonella typhimurium, Listeria monocytogenes and Listeria innocua are used in the method according to the invention.

The glycerol dehydratase used is preferably coded by the genes selected from the group consisting of pduC, pduD, pduE, pddA, pddB, pddC, dhaB, dhaC, dhaE and the gldABC genes from Lactobacillus reuteri ATCC55730. Also advantageous is the use of the glycerol dehydratase variant SHGDH22 described in WO-A-2004/056963 which has the SEQ.-ID No. 322 disclosed in WO 2004056963. The nucleotide sequence of this and further suitable genes for a glycerol dehydratase can be found, for example, in the “Kyoto Encyclopedia of Genes and Genomes” (KEGG database), the databases of the National Center for Biotechnology Information (NCBI) of the National Library of Medicine (Bethesda, Md., USA) or the nucleotide sequence database of the European Molecular Biologies Laboratories.

In connection with the diol dehydratase, it may be advantageous to use in the method in particular the diol dehydratase which can be isolated from microorganisms selected from the group of the genera Klebsiella, Propionibacterium, Clostridium, Lactobacillus, Salmonella, Citrobacter, Flavobacterium, Acetobacterium, Brucella and Fusobacterium, particularly preferably can be isolated from microorganisms selected from the group of the species Klebsiella pneumoniae, Propionibacterium freudenreichii, Clostridium glycolicum, Lactobacillus brevis, Salmonella typhimurium, Citrobacter freundii, Lactobacillus buchneri, Brucella melitensis, Fusobacterium nucleatum, Klebsiella oxytoca, Salmonella typhimurium, Listeria monocytogenes and Listeria innocua.

In connection with the propanediol dehydratase, it may be advantageous in the method in particular the propanediol dehydratase which can be isolated from microorganisms selected from the group of the genera Citrobacter, Clostridium, Klebsiella, Lactobacillus, Propionibacterium and Salmonella, particularly preferably can be isolated from microorganisms selected from the group of the species Citrobacter freundii, Clostridium glycolicum, Klebsiella oxytoca, Klebsiella pneumoniae, Lactobacillus brevis, Lactobacillus buchneri, Lactobacillus collinoides, Propionibacterium freudenreichii and Salmonella typhimurium.

In the case where the hydro-lyase used in the method according to the invention is a vitamin B₁₂ dependent enzyme, it may be advantageous to carry out step A) in the presence of an enzyme and/or enzyme complex having glycerol dehydratase reactivase activity or diol dehydratase reactivase activity since the hydro-lyase can possibly be deactivated through binding of the vitamin. Such a reactivase is described for example in Mori et al., J. Biol. Chem. 272:32034 (1997). Preference is given to using reactivases which can be isolated from the organism from which the corresponding hydro-lyase can be isolated. Suitable diol dehydratase reactivases are known from Klebsiella oxytoca (GenBank Nos: AAC15871, AF017781; GenBank Nos: AAC15872, AF017781) Salmonella typhimurium (GenBank Nos: AAB84105, AF026270; GenBank Nos: AAD39008, AF026270) and Lactobacillus collinoides (GenBank Nos: CAD01092, AJ297723; GenBank Nos: CAD01093, AJ297723), suitable glycerol dehydratase reactivase from Klebsiella pneumoniae (cf. WO 2008137403).

The hydro-lyases used according to the invention in the method can be used in all forms of accessibility known to the person skilled in the art. Thus, the hydro-lyases used can be used as isolated enzymes, isolated for example from a recombinant expression system, or as a constituent of enzyme complexes. Preference is given to using the hydro-lyases in the form of a whole-cell reactor, i.e. the hydro-lyases are present in a biological cell, preferably in the cell which expresses the hydro-lyase.

The cells having hydro-lyase can express the hydro-lyase already as wild type or can have been genetically modified such that they only have a hydro-lyase activity as a result of the genetic modification.

Wild type cells containing hydro-lyase which can be used in the method according to the invention are the cells which are described above as microorganisms from which the hydro-lyases can be isolated.

Genetically modified cells which contain suitable hydro-lyase and can be used in the method according to the invention are described, for example, in WO 2008148640, to the disclosure of which reference is explicitly made here.

Furthermore, it may be advantageous to reduce the tendency of the cells towards the reactive formation of by-products from the aldehydes, in particular the tendency of the corresponding aldehydes to reduce to the corresponding alcohols, through appropriate genetic modification of the cells. For example, in cases where the aldehyde to be produced by the method according to the invention is 3-hydroxypropionaldehyde, cells with an activity, that is reduced compared to their wild type, of an enzyme E₁ which is able to reduce 3-hydroxypropionaldehyde to 1,3-propanediol are used. Enzyme E₁ (1,3-propanediol-dehydrogenase, 3-hydroxypropionaldehyde reductase, 1,3-propanediol:NAD⁺ oxidoreductase or propane-1,3-diol:NAD⁺-1-oxidoreductase; EC 1.1.1.202) catalyses the reaction 3-hydroxypropionaldehyde+NAD(P)H→1,3-propanediol+NAD(P)⁺. Such enzymes are e.g. the proteins with the GenBank accession numbers AP007281.1, EDX43365.1, BAG24545.1, ABQ82306.1, ABO43839.1, EEI08979.1, EEI166346.1, EEI73647.1, EEJ92522.1, EEI09194.1, EEI73500.1, ABQ83973.1 or BAG26138.1.

From this data it is possible to identify corresponding oxidoreductase for the cell containing the particular hydro-lyase which is used in the method according to the invention and to reduce its activity compared to its wild type.

The wording “reduced activity of an enzyme” used is preferably understood as meaning an activity reduced by a factor of at least 0.5, particularly preferably of at least 0.1, moreover preferably of at least 0.01, moreover even more preferably of at least 0.001 and most preferably of at least 0.0001. The reduction in the activity of a certain enzyme can take place, for example by targeted or undirected mutation, by the addition of competitive or non-competitive inhibitors or by other measures, known to the person skilled in the art, for reducing the synthesis of a specific enzyme.

Of suitability for reducing the activity of an enzyme E₁ by targeted mutation are, for example, directed knockouts, in which, for example, promoter active sequences of the target gene, active regions (necessary for the catalytic function of the enzyme) of the target gene or the entire target gene are deleted, substituted with foreign DNA or are interrupted with foreign DNA.

Furthermore, various techniques of gene silencing are known to the person skilled in the art which, via antisense nucleic acids or small-interfering RNA (siRNA), reduce, or even completely prevent, specifically the transcription and/or the translation of individual genes. Depending on the selected system, this suppression can take place transiently, can be inducible or be present constitutively.

Methods are likewise known to the person skilled in the art with which individual point mutations can be placed in the target gene in a targeted manner. Furthermore, methods are known to the person skilled in the art with which mutations can be placed in the target gene in an undirected manner in order to then select cells which have a reduced activity of an enzyme E₁. Thus, such mutations can be triggered e.g. by the natural error rate during the replication of genetic material or by exposing the cells to physical or chemical noxae, such as, for example, UV light, N-methyl-N′-nitro-N-nitrosoguanidine, ethyl methanesulfonate, nitrose acid, hydroxyamine or 4-nitroquinoline 1-oxide.

The cells containing hydro-lyase used according to the invention in the method may be living cells or dead cells, preference being given to living cells.

It may be advantageous for the living hydro-lyase-containing cells used as hydro-lyases to be kept in a suitable medium during the method, said medium supplying them with the substances required for growth. However, it has been found that, as regards supply with oxygen, it may be advantageous to limit this. Consequently, it is preferred for the living, hydro-lyase-containing cells used as hydro-lyases to be used in step A) under microanaerobic or anaerobic conditions. Microanaerobic or anaerobic conditions are preferably characterized in that the medium surrounding the living, hydro-lyase-containing cells used as hydro-lyases has a content of dissolved oxygen of less than 1 mg/L, preferably of less than 0.5 mg/L, in particular of less than 0.1 mg/L, based on the total medium. These conditions can be achieved, for example, by the medium surrounding the cells not being additionally gassed by air or oxygen, i.e. air or oxygen is not passed through the medium, and, if appropriate, additionally the container surrounding the reaction mixture is sealed as far as possible in an air-tight manner.

In order to be able to carry out the separation of aldehyde and hydro-lyase to be undertaken in step B) in a simplified manner, it is preferred for the hydro-lyase to be immobilized on a support. If the hydro-lyase is used as free, isolated enzyme, many methods of enzyme immobilization are available to the person skilled in the art, such as, for example, the coupling to epoxide-activated support materials and methods described in the literature, such as, for example: Mateo et al. Advances in the design of new epoxy supports for enzyme immobilization-stabilization, Biochem Soc Trans. 2007 December; 35(Pt 6):1593-601 and Balcão et al., Bioreactors with immobilized lipases: state of the art, Enzyme Microb Technol. 1996 May 1; 18(6):392-416.

If cells having hydro-lyase activity are used as hydro-lyases, methods known to the person skilled in the art are used for immobilizing the cells, such as, for example, calcium alginate methods. In particular, the siloxane-based method described in DE 102007031689, and also the immobilization of the cells with the commercially available Lentikat liquid is particularly suitable in this connection.

In the method according to the invention, substituted and unsubstituted vicinal diols can be used in step A); preferably, the substituents are selected from the group comprising carboxyl, amino, ester, isocyanate and hydroxyl groups, where OH is particularly preferred.

In the method according to the invention, use is made preferably of vicinal diols with a carbon chain length of from 2 to 20, in particular of from 2 to 12 and very particularly of from 2 to 6 carbon atoms. Preferred vicinal diols used in the method according to the invention include in particular sugars and sugar alcohols having 3 to 6 carbon atoms, such as, for example, sorbitol; particular preference is given in this connection to 1,2-propanediol, ethylene glycol and glycerol.

In particular, glycerol is used in the method according to the invention as vicinal diol, meaning that the resulting aldehyde is 3-hydroxypropionaldehyde. As well as glycerol, in particular also 1,2-propanediol is used, in which case propanal is obtained as aldehyde. This aldehyde can be converted to n-propanol and propionic acid through a disproportionation, which readily follows.

The vicinal diol is used in step A) in a concentration range from 0.01% by weight to 50% by weight, preferably from 0.2% by weight to 20% by weight, based on the total reaction mixture.

In step B), the aldehyde formed in step A) and the hydro-lyase are separated.

The separation of the hydro-lyase, immobilized in particular on a support, takes place here by means of separation methods known to the person skilled in the art, such as, for example, centrifugation. If hydro-lyase-containing cells are used as hydro-lyase in step A), it is conceivable to separate the cells from the aldehyde for example by means of a suitable filter, for example by means of a filter with an exclusion size in a range from 20 to 200 kDa. Also conceivable is the use of a centrifuge, of a suitable sedimentation device or of a combination of these devices, it being particularly preferred to separate off at least some of the cells firstly by sedimentation and then to pass the aldehyde, partially freed from the cells, to an ultrafiltration or centrifugation device.

The cells separated off in this way, if they have been obtained in several separation stages carried out in succession, are optionally purified and can be passed directly again to step A). However, it may prove to be advantageous to again wash the hydro-lyase-containing cells before carrying out step A) again, in which case this washing preferably takes place with the same medium which is used in step A).

Particularly preferably, the washing with the medium used in step A) takes place with the additional addition of glucose; by doing so, the formation of by-products derived from the aldehyde can be suppressed in an advantageous manner during the actual biotransformation.

For washing the cells, these can, for example, be resuspended in a suitable volume of the medium used in step A) and then be freed from the medium by the separation methods described above, in particular by filtration, sedimentation or centrifugation, or else by a combination of these measures.

Analogous washing operations can likewise be used for hydro-lyases immobilized on a support.

Similarly, the separation of aldehyde and hydro-lyase to be undertaken in step B) can be made easier by immobilizing the compound which can form a bond with the aldehyde, on a support. Of suitability here are, for example, ion exchanger resins laden with hydrogen sulfite, such as Amberlite® CG400 or Amberlite® IRA400 (Rohm and Haas).

It is essential to the invention that step A) and B) is repeated at least once with the separated-off hydro-lyase in order to utilize the biocatalyst again with fresh vicinal diol to produce the aldehyde.

In a preferred, alternative embodiment of the method according to the invention, steps A), B) and C) are carried out continuously.

This can be realized in different ways:

The hydro-lyase is present in immobilized form as stationary phase, and a medium containing the vicinal polyol and the compound which forms a bond with the aldehyde flows around the immobilized hydro-lyase and, according to step A), is brought into contact with it. The resulting aldehyde is separated off as mobile phase from the stationary phase, which corresponds to the separation of aldehyde and hydro-lyase in step B). The vicinal diol continuously fed afresh to the hydro-lyase corresponds to a repetition of steps A) and B) with the hydro-lyase obtained from step B).

Alternatively, the compound which forms a bond with the aldehyde can be present in immobilized form as a stationary phase. The vicinal polyol and the hydro-lyase are present in a medium and thus, according to step A), brought into contact and flow around the immobilized compound which forms a bond with the aldehyde, in which case the aldehyde formed remains on the stationary phase. The hydro-lyase leaves the stationary phase, which corresponds to the separation of aldehyde and hydro-lyase in step B), and is fed to the stationary phase again, together with fresh vicinal polyol, for example in the form of a loop reactor. A further variant of a method according to the invention which is operated continuously is designed such that both the compound which forms a bond with the aldehyde and also the hydro-lyase are present in immobilized form as stationary phases. A medium containing the vicinal polyol flows around the stationary phase and is thus brought into contact with the hydro-lyase according to step A). The aldehyde formed is bonded to the stationary phase by the compound which forms a bond with the aldehyde and has thus been separated from the hydro-lyase according to step B). The vicinal diol continuously fed afresh to the stationary phases corresponds to a repetition of steps A) and B) with the hydro-lyase obtained from step B).

In order to achieve the set object, a compound which forms a bond with the aldehyde must be present during the method according to the invention.

The bond formed with the aldehyde can be any known form of chemical bond, such as, for example, covalent or ionic bonds, but also the so-called weak bonds such as hydrogen bridge bonds or bonds brought about by electrostatic interactions.

Preferably, the compound which forms a bond with the aldehyde forms a covalent bond with the aldehyde.

The compound which forms a bond with the aldehyde can be polyfunctional, i.e. more than one bond to the aldehyde can be formed per molecule of compound.

Suitable compounds which form a bond with the aldehyde are, for example, hydrazides, amines, hydrazines, urea compounds, hydroxylamines, alcohols, mercaptans, hydrogensulfites, sulfites, metabisulfites and pyrosulfites, where, in the method according to the invention, preference is given to using semicarbazide [CAS 57-56-7], carbohydrazide [CAS 497-18-7] and sodium sulfite [CAS 7757-83-7].

These can be used free in solution or else immobilized on supports, preference being given in this connection to non-immobilized, dissolved compounds since these can enter into a bond with the aldehyde more effectively and more quickly and are thus able to prevent undesired secondary reactions.

If the method according to the invention is carried out with cells, it is preferred according to the invention if the compound which forms a bond with the aldehyde is added to the medium surrounding the cells.

Mixtures of the compounds which form a bond with the aldehyde can also be used.

It is preferred if, in the method according to the invention, the vicinal diol and the functional group of the compound which forms a bond with the aldehyde are used in a molar ratio of at least 1:1, preferably of at least 1:1.2, in particular of at least 1:1.5.

In step D) of the method according to the invention, the aldehydes formed can be isolated where, for the isolation, all methods known to the person skilled in the art for the isolation of low molecular weight substances from complex compositions are suitable. By way of example, mention may be made at this point of precipitation by means of suitable solvents, extraction by means of suitable solvents, complexation, for example by means of cyclodextrins or cyclodextrin derivatives, crystallization, purification and/or isolation by means of chromatographic methods or conversion of the aldehydes to derivatives which can be separated off easily.

Part of the isolation in step D) can also be to separate the compound which forms a bond with the aldehyde again from the aldehyde.

This can be achieved by methods known per se to the person skilled in the art, depending on the bond present. Thus, for example, bonds between aldehydes and semicarbazide or carbohydrazide can be cleaved by acid; bonds between the complex of aldehyde and the compound entering into a bond with the aldehyde, and also Amberlite®CG400 or Amberlite® IRA400 can be separated, for example, by increasing the salt concentrations, for example of 1M NaCl or a system of NaHCO₃/Na₂CO₃ (0.15 M/0.075 M). Furthermore, a bond between the aldehyde and the compound which enters into a bond with the aldehyde can be broken by a thermal treatment.

A further contribution to achieving the object specified at the start is also made by a method for producing oxidation or reduction products of an aldehyde, comprising the steps:

I) provision of an aldehyde by means of the method described above for producing aldehydes from vicinal diols,

II) chemical or biocatalytic reaction of the aldehyde by reduction, oxidation, disproportionation or dehydration to give optionally unsaturated reduction or oxidation intermediates and optionally

III) the further chemical or biocatalytic reaction of the reduction or oxidation intermediates obtained in step II) by reduction, oxidation, or addition.

Oxidation or reduction products of the aldehyde include compounds such as, for example, optionally unsaturated carboxylic acids, carboxylic acid derivatives, amines, isocyanates, acetals, nitriles and oximes.

The reduction or oxidation intermediates of step II) can therefore already be the desired reduction or oxidation products of the aldehyde.

Firstly, in step I), an aldehyde is provided by means of the method described above for producing aldehydes, where said aldehyde can optionally, as described above in connection with this method, have been purified.

In step II), the aldehyde can then be converted chemically or biocatalytically by reduction, oxidation, disproportionation or dehydration, to give reduction or oxidation intermediates, particularly if the aldehyde obtained in step I) is 3-hydroxypropionaldehyde, to give 1,3-propanediol (1,3-PDO) (reduction), acrolein (dehydration) or 3-hydroxypropionic acid (oxidation). In this connection, details relating to the conversion of 3-hydroxypropionaldehyde to acrolein are described, inter alia, by Hall and Stern in Journal of the Chemical Society, 1950, pages 490-498, whereas details on producing 1,3-propanediol from 3-hydroxypropionaldehyde can be found inter alia in U.S. Pat. No. 5,334,778. The production of 3-hydroxypropionic acid from 3-hydroxypropionaldehyde in turn is described, inter alia, in U.S. Pat. No. 6,852,517. If the aldehyde obtained in step I) is propanal from 1,2-propanediol, then 1-propanol and propionic acid can be formed by ready disproportionation of the propanal in the biotransformation medium (Sriramulu, D. D.; Liang, M.; Hernandez-Romero, D.; Raux-Deery, E.; Lunsdorf, H.; Parsons, J. B.; Warren, M. J. & Prentice, M. B. J. Bacteriol., 2008, 190, 4559-4567).

If appropriate, the reduction or oxidation intermediates obtained in step II) can be converted yet further by chemical or microbiological means by reduction, oxidation or addition in a further step III). Of suitability here is in particular the conversion of acrolein obtained in step II) by oxidation to give acrylic acid, which can then, if appropriate, be converted in a yet further step IV) by radical means with the formation of polymers based on acrylic acid. Of suitability here is in particular the radical polymerization of optionally partially neutralized acrylic acid in the presence of suitable crosslinkers to form water-absorbing polyacrylates, which are also referred to as “superabsorbents”. The chemical oxidation of acrolein to acrylic acid and the subsequent radical polymerization of the acrylic acid obtained in this way is described, inter alia, in WO-A-2006/136336.

Furthermore, it is likewise contemplated that the conversion of acrolein obtained in step II) by oxidative esterification to acrylic acid esters, which can then, if appropriate, be converted in a yet further step IV) by radical means with the formation of polymers based on acrylic acid esters.

Preferred reduction or oxidation products of the aldehyde obtainable from the method according to the invention via steps explained above are thus the substances 1,3-propanediol, 1-propanol, acrolein, 3-hydroxypropionic acid, propionic acid, acrylic acid, acrylic acid esters and polymers based on acrylic acid or acrylic acid esters, where the aldehyde obtained in step I) is 3-hydroxypropionaldehyde.

In the examples listed below, the present invention is described by way of example without any intention of limiting the invention, the scope of application of which arises from the entire description and the claims, to the embodiments specified in the examples.

The following figures are part of the examples:

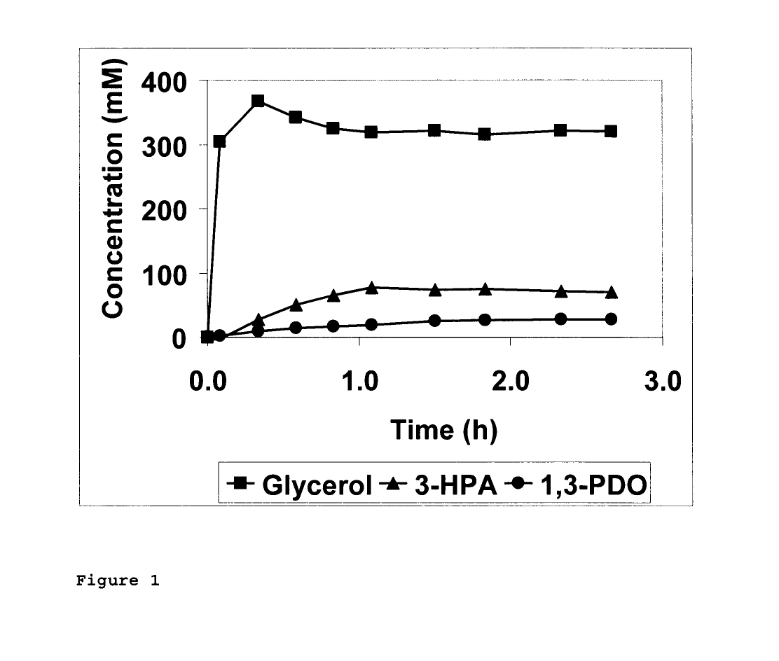

FIG. 1: concentration course of glycerol, 3HPA and 1,3-PDO during conversion of glycerol with immobilized L. reuteri cells in the course of one biotransformation cycle.

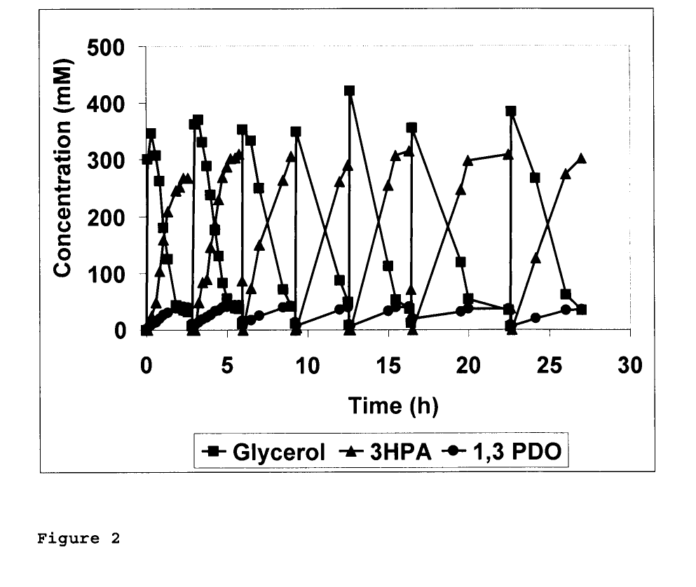

FIG. 2: concentration course of glycerol, 3HPA and 1,3-PDO during conversion of glycerol with immobilized L. reuteri cells in the course of 7 biotransformation cycles and in the presence of semicarbazide as scavenger.

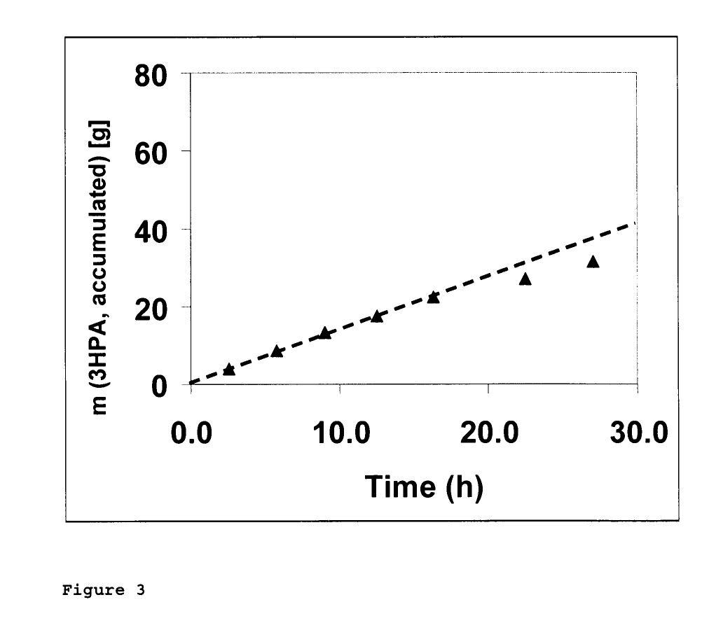

FIG. 3: accumulated yield of 3HPA during conversion of glycerol with immobilized L. reuteri cells in the course of 7 biotransformation cycles and in the presence of semicarbazide as scavenger.

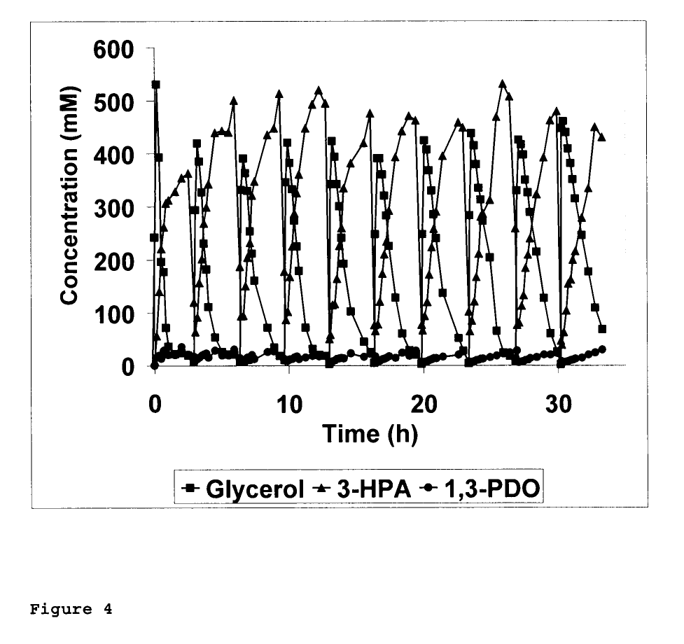

FIG. 4: concentration course of glycerol, 3HPA and 1,3-PDO during conversion of glycerol with immobilized L. reuteri cells in the course of 10 biotransformation cycles and in the presence of carbohydrazide as scavenger.

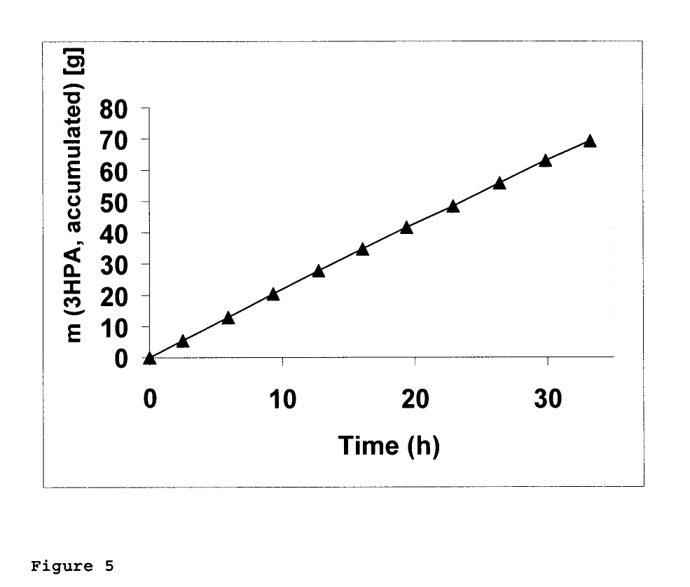

FIG. 5: accumulated yield of 3HPA during conversion of glycerol with immobilized L. reuteri cells in the course of 10 biotransformation cycles and in the presence of carbohydrazide as scavenger.

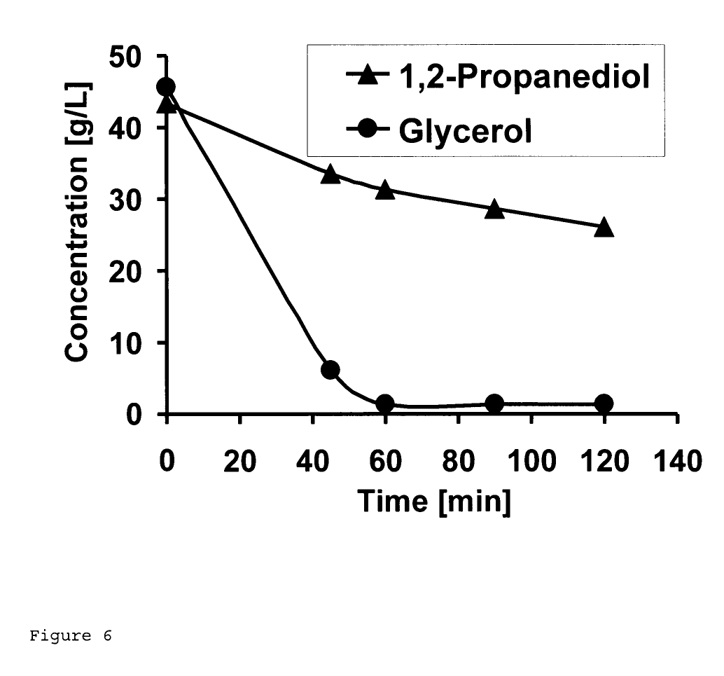

FIG. 6: course with respect to time of the concentrations of glycerol and 1,2-propanediol during biotransformation of glycerol and of 1,2-propanediol with L. reuteri cells.

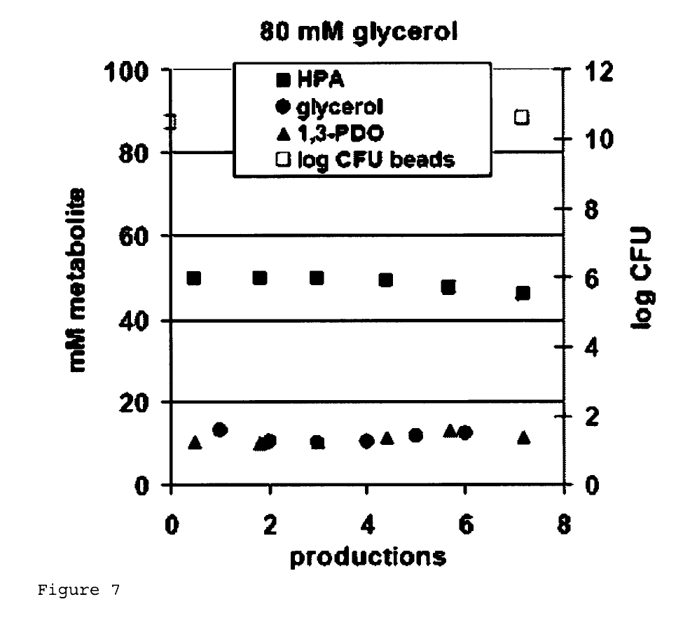

FIG. 7: course of the concentrations of 3HPA, glycerol and 1,3-PDO during seven successive biotransformations of glycerol with immobilized L. reuteri cells without the addition of sulfite (comparative example)

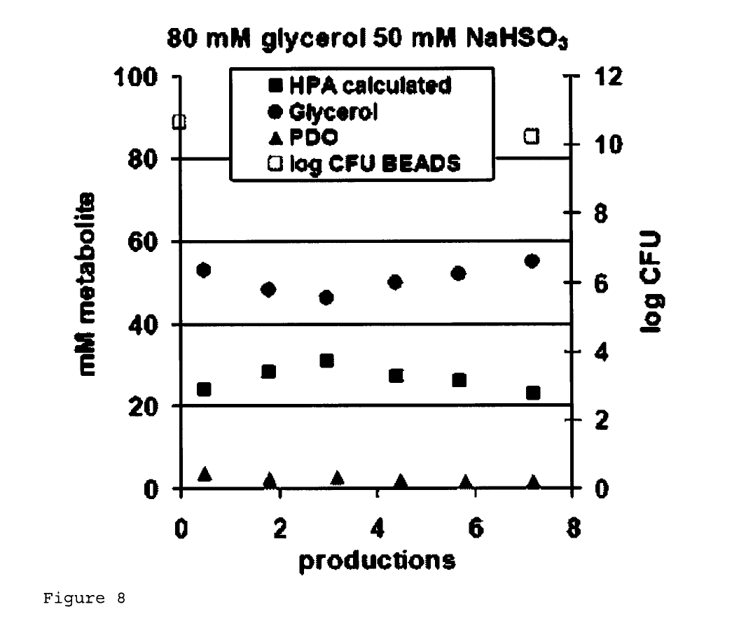

FIG. 8: course of the concentrations of 3HPA, glycerol and 1,3-PDO during seven successive biotransformations of glycerol with immobilized L. reuteri cells with the addition of sodium hydrogensulfite (according to the invention).

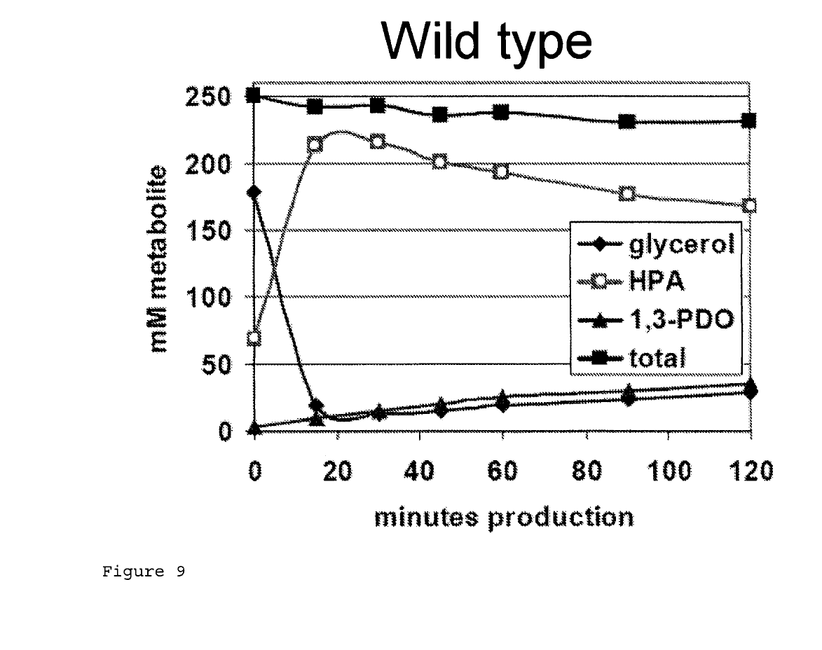

FIG. 9: course with respect to time of the concentrations of 3HPA, glycerol and 1,3-PDO during biotransformation of glycerol with wild type L. reuteri cells.

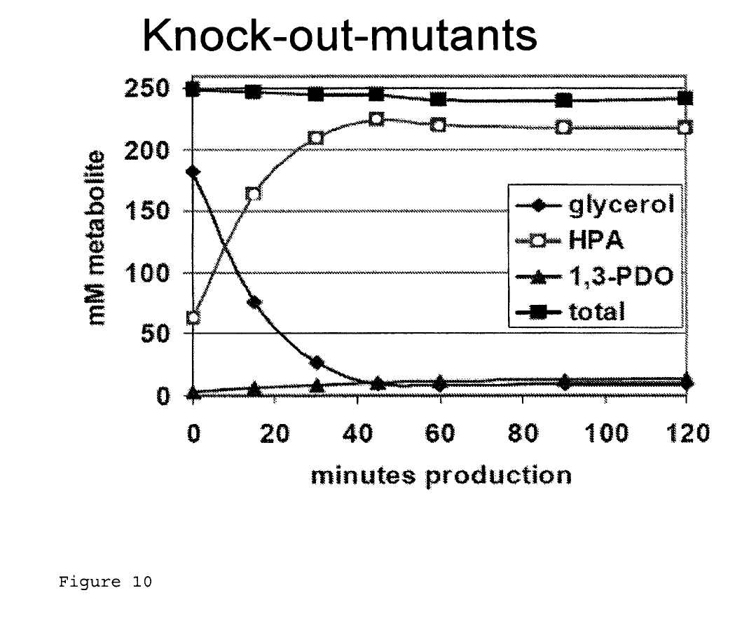

FIG. 10: course with respect to time of the concentrations of 3HPA, glycerol and 1,3-PDO during biotransformation of glycerol with genetically modified L. reuteri cells.

### EXAMPLES

### Example 1

### Cultivation of Lactobacillus reuteri

The cultivation of Lactobacillus reuteri was carried out in anaerobicized MRS medium (medium for Lactobacillus according to DeMan, Rogosa and Sharpe) with the addition of 20 mM of glycerol (MRSG20) in closed suitable packs at 80 rpm. The medium MRSG20 (composition [data per liter]: 10 g of proteose peptone No. 3 (Roth), 8 g of beef extract (Roth), 4 g of yeast extract (VWR), 1.5 mL of glycerol (Roth), 1 mL of Tween80 (Roth), 40 mL of salt solution [10 g/L of dipotassium hydrogenphosphate {Roth}, 250 g/L of sodium acetate trihydrate {Roth}, 100 g/L of diammonium hydrogen citrate {Roth}, 10 g/L of magnesium sulfate heptahydrate {Roth}, 2.5 g/L of manganese sulfate tetrahydrate {Roth}], following anaerobicization and autoclaving (20 min at 121° C.), was admixed with 25 mL of a glucose solution (4 mol/L). For preculture 1 (50 mL), a cryo culture was used. For this, L. reuteri SD2112 was stored in MRS medium, which additionally comprises 6% (w/v) of low-fat milk powder and 10% (v/v) of glycerol, at −80° C. To produce the stock culture, an overnight culture (10 ml of MRS medium) is mixed 1:1 with the stock solution. The preculture served as 1% strength inoculum and was incubated at 33° C. After 15 hours, from this batch, a second preculture (50 mL) was inoculated to 1% strength and incubated for 9 hours at 37° C. This served in turn as 1% strength inoculum for the main culture (2 times 1 L). After 15 hours at 33° C., the cells were harvested.

For this, 2 liters were centrifuged off (4000 g; 20° C.; 10 min), the supernatant was discarded and both resulting pellets were resuspended in 1 liter 0.1 M potassium phosphate buffer (KPP; K₂HPO₄+KH₂PO₄ [Roth]) at pH 7. Centrifugation was carried out again under the same conditions. The supernatant was again discarded, the cell pellet was resuspended in 0.1 M KPP at pH 7 in the ratio 1 g to 1 mL and stored under N₂. This suspension was used for the subsequent immobilization.

### Example 2

### Immobilization of Lactobacillus reuteri

For the immobilization, 20 mL of the cell suspension described in example 1 were mixed with Lentikat-Liquid® (GeniaLab) heated to ˜35° C., the Lentikats were produced with the LentiKat®-Printer (GeniaLab) and dried to a residual moisture of 28%. After back-swelling in LentiKat-Stabilizer® (GeniaLab), the immobilizates were stirred for at least 2 hours in anoxic atmosphere at 300 rpm to stabilize the Lentikat in LentiKat-Stabilizer®. The immobilizates separated-off and washed twice with 0.1 M KPP at pH 7 were regenerated to 3% strength for 15 hours at 33° C. standing in MRSG20 in a completely filled 1 L bottle with the option for gas exchange. After separating the immobilizates from the medium (aerobic conditions), these were washed twice with 0.1 M KPP (pH 7).

### Example 3

### HPLC-Based Quantification of Glycerol and 1,3-propanediol

To quantify glycerol and 1,3-propanediol (1,3-PDO), in each 1 ml of cell suspension, the biomass was separated off by centrifugation (10 min, 16.100 g, 4° C.) and 40 μl of the culture supernatant were analyzed using a Shimadzu HPLC system (Autosampler SIL-10AT, Pump LC-10AT, Degasser DGU-3A, refractive index detector RID-10A, UV detector SPD-10A, oven CTO-10A, Controller SCA-10A-VP) with an Aminex HPX-87H 300×7.8 mm separating column (Bio-Rad Laboratories GmbH, Munich) with a particle size of 9 μm and using a precolumn (HPX-87H, 30×4.6 mm). The substances to by analyzed were eluted isocratically with a mobile phase from 5 mM of sulfuric acid with a flow rate of 0.6 ml/min for 20 min at 40° C. Identification was made on the basis of a comparison with the residence time of reference substances (Sigma-Aldrich Chemie GmbH, Steinheim). The concentration of the compounds in the culture supernatants was calculated via a comparison of the peak areas with a calibration curve generated beforehand with external standards.

### Example 4

### Conversion of Glycerol to 3HPA with Immobilized Lactobacillus reuteri (Comparative Example)

The immobilizates prepared as in example 2 and laden with L. reuteri were used at 10% strength (20 g, corresponding to a concentration of 2 g of dry cells per liter) in a biotransformation in a glass fermenter (total volume 500 mL) with 200 mL of reaction buffer. The latter consisted of anaerobicized and preheated (to 35° C.) 0.1 M KPP (pH 7). After establishing a constant pH of 7 and a constant temperature of 35° C., the reaction was started by adding 8 mL of 98% strength glycerol (Roth, corresponding to a concentration of 500 mM in the reaction mixture).

Sampling was carried out every 10 minutes for one hour and then every 30 minutes for 2 hours. The concentrations of glycerol and of the by-products were determined by means of HPLC as in example 3.

Quantification of 3-hydroxypropionaldehyde was carried out by means of a colorimetric test as in Doleyres et al. (“Production of 3-hydroxypropionaldehyde using a two-step process with Lactobacillus reuteri, Applied Microbiology and Biotechnology, Vol. 68, 2005, pages 467-474).

After a reaction time of −100 minutes, conversion of glycerol could no longer be observed both in the case of the free cells and also in the case of the immobilized cells.

Concentration courses for glycerol, 3HPA and 1,3-PDO are given in FIG. 1; during the biotransformation, 1.29 g of 3HPA were produced. The reaction medium was separated off from the cells and immobilizates and stored separately at 4° C.

The conversion of glycerol to 3HPA and the by-products took place, according to the analysis described above, to −25%. Upon re-use of the cells and/or immobilizates, no conversion of glycerol to 3HPA could be established.

### Example 5

### Conversion of Glycerol to 3HPA with Immobilized Lactobacillus reuteri and in the Presence of Semicarbazide, According to the Invention

The immobilizates produced as in example 2 and laden with L. reuteri were used at 10% strength (20 g, corresponding to a concentration of 2 g of dry cells per liter) in a biotransformation in a glass fermenter (total volume 500 mL) with 200 mL of reaction buffer. The latter consists of 0.1 M KPP (pH 7) and also 500 mM semicarbazide and has been anaerobicized and also preheated to a reaction temperature of 35° C. After establishing a constant pH of 7 and a constant temperature of 35° C., the reaction was started by adding 8 mL of 98% strength glycerol (Roth; corresponding to a concentration of 500 mM in the reaction mixture).

The removal and analysis of samples from the reaction mixture was carried out as described in example 4.

After a reaction time of 150 minutes, the reaction medium was separated off from the immobilizates and fresh anaerobicized, preheated reaction medium (0.1 M KPP pH 7 with 500 mM of semicarbazide) was added. As described above, a further biotransformation cycle was carried out. In total, 7 successive cycles without interim storage of the immobilizates were carried out. Concentration courses for glycerol, 3HPA and 1,3-PDO are shown in FIG. 2; FIG. 3 shows the accumulated yields of 3HPA.

The conversion of glycerol to 3HPA took place with semicarbazide as scavenger, according to the analysis described above, in the first 5 cycles only to ˜55%; in the 2 further cycles, the yield was continuously reduced.

### Example 6

### Conversion of Glycerol to 3HPA with Immobilized Lactobacillus reuteri in the Presence of Carbohydrazide, According to the Invention

The immobilizates produced as in example 2 and laden with L. reuteri were used at 10%, strength (20 g, corresponding to a concentration of 2 g of dry cells per liter) in a biotransformation in a glass fermenter (total volume 500 mL) with 200 mL of reaction buffer. The latter consists of 0.1 M KPP (pH 7) and also 520 mM of carbohydrazide and has been anaerobicized and also preheated to a reaction temperature of 35° C. After establishing a constant pH of 7 and a constant temperature of 35° C., the reaction was started by adding 8 mL of 98% strength glycerol (Roth; corresponding to a concentration of 500 mM in the reaction mixture).

The removal and analysis of samples from the reaction mixture was carried out as described in example 4.

After a reaction time of 180 minutes, the reaction medium was separated off from the immobilizates and fresh anaerobicized, preheated reaction medium (0.1 M KPP pH 7 with 520 mM of carbohydrazide) was added. As described above, a further biotransformation cycle was carried out. In total, 10 successive cycles without interim storage of the immobilizates were carried out; concentration courses for glycerol, 3HPA and 1,3-PDO are shown in FIG. 4; FIG. 5 shows the accumulated yields of 3HPA.

The conversion of glycerol to 3HPA and by-products took place with carbohydrazide as scavenger, according to the analysis described above, in all 10 cycles to ˜95%.

### Example 7

### Conversion of Glycerol to 3HPA with Free Lactobacillus reuteri in the Presence of Sulfur-Containing Compounds, According to the Invention

The cells produced as in example 1 were suspended in a 1000 millimolar glycerol solution such that an overall cell concentration of 2.2×10¹⁰ CFU/ml results. After adding a sulfur-containing compound (sodium sulfite, sodium hydrogensulfite or sodium pyrosulfite; in each case with an end concentration of 100 mmol/L in the reaction solution), the suspension was incubated for 120 min, during which the mixture was kept oxygen-free by introducing a continuous stream of nitrogen. The concentrations of 3HPA and 1,3-PDO resulting after 120 min, and also the cell viabilities, listed in table 1, show the increase in the achievable 3HPA concentration and also the reduction in the cell viability decrease as a result of adding the sulfur-containing compounds.

TABLE 1

| Sulfur-containing | c (3-HPA) | c (1,3-PDO) | Cell viability |
|---|---|---|---|
| compound | [mmol/L] | [mmol/L] | [CFU/mL] |
| None | 504 | 32 | 5.1*10⁴ |
| Na₂S₂O₅ | 524 | 31 | >6.0*10⁹ |
| NaHSO₃ | 546 | 30 | 3.2*10⁹ |
| Na₂SO₃ | 607 | 31 | 2.6*10⁹ |

### Example 8

### Conversion of 1,2-propanediol or Glycerol to Propanal or 3-hydroxypropionaldehyde, Respectively, with Immobilized Lactobacillus reuteri in the Presence of Carbohydrazide, According to the Invention

The cells produced as in example 1 were in each case suspended in a 500 millimolar glycerol solution and/or a 500 millimolar 1,2-propanediol solution such that an overall cell concentration of 2.2×10¹⁰ CFU/ml results. After adding carbohydrazide with an end concentration of 100 mmol/L in the reaction solution, the suspension was incubated for 120 min, during which the mixture was kept oxygen-free by introducing a continuous stream of nitrogen. The concentration courses of glycerol and 1,2-propanediol shown in FIG. 6 illustrate the equivalent transformation of the two substrates.

### Example 9

### Conversion of Glycerol to 3HPA with Immobilized Lactobacillus reuteri Cells in the Presence of Sulfur-Containing Compounds and with the Addition of Glucose to the Washing Solution, According to the Invention

The immobilizates produced as in example 2 and laden with L. reuteri were used at 20% strength (4 mL) in a biotransformation in a glass fermenter with 20 mL of reaction solution. The latter comprises 80 mmol/L of glycerol and, after heating to 30° C., was adjusted to a constant pH of 5 (comparative example and example according to the invention). In the mixture according to the invention, sodium hydrogensulfite was additionally added to the reaction solution (according to an end concentration of 50 mmol/L). Following completion of one biotransformation cycle (30 min), the immobilizates were filtered off, regenerated for a period of 30 min in MRSG20 (see example 1) and used again as described in a reaction solution. In the manner described, a total of 7 conversions of glycerol are carried out. Whereas without the addition of hydrogensulfite in each transformation step, 10 mmol/L of 1,3-PDO are formed (FIG. 7), when adding sodium hydrogensulfite, a reduction in the 1,3-PDO concentration can be observed (FIG. 8).

### Example 10

### Conversion of glycerol to 3HPA with free, Genetically Unmodified or Modified Lactobacillus reuteri, According to the Invention

The wild type cells or the cells generated by deletion of the oxidoreductase gene were suspended in a 250 millimolar glycerol solution such that in each case an overall cell concentration of 1.2×10¹⁰ CFU/ml results. As the concentration courses in the course of a 120 minute biotransformation show, when using the L. reuteri wild type, the 3HPA formed after 20 minutes' biotransformation is degraded in favor of the formation of 1,3-PDO (FIG. 9), whereas the conversion of 3HPA to 1,3-PDO is prevented when using the cells generated by deletion of the oxidoreductase gene (FIG. 10).

### Example 11

### Production of Acrolein from a Fermentation Product Containing 3HPA, According to the Invention

It was possible to obtain acrolein from the fermentation products obtained as in examples 4-6 by acidic hydrolysis at elevated temperatures. For this, some of the fermentation product was diluted with water in the ratio 1:100, mixed with 3 parts of 37% strength hydrochloric acid and incubated for 0.5 h at 37° C. Following addition of a DL-tryptophan solution (Fluka, 0.01 mol/L of tryptophan in 0.05 mol/L of hydrochloric acid) and incubation a further time at 37° C. (0.5 h), the acrolein which had formed in the process formed a colored adduct, which could be quantified by colorimetry through its absorption at 560 nm.

1. A method for producing an aldehyde, comprising
A) contacting a vicinal diol with a hydro-lyase,
B) separating an aldehyde formed in A) from the hydro-lyase,
C) repeating A) and B) at least once with the hydro-lyase obtained from B), and
D) optionally isolating the aldehyde,
wherein the method is carried out in the presence of a compound which forms a bond with the aldehyde.

2. The method of claim 1, wherein the hydro-lyase in A) is a glycerol dehydratase.

3. The method of claim 1, wherein the hydro-lyase in A) is immobilized on a support.

4. The method of claim 1, wherein the hydro-lyase is present in cells having hydro-lyase activity.

5. The method of claim 4, wherein the cells have an activity, which is reduced compared to their wild type, of an oxidoreductase which is able to reduce the aldehyde.

6. The method of claim 1, wherein the vicinal diol in A) is glycerol or 1,2-propanediol.

7. The method of claim 1, wherein the vicinal diol in A) is used in a concentration range from 0.01% by weight to 50% by weight based on the total reaction mixture.

8. The method of claim 1, wherein A), B) and C) are carried out continuously.

9. The method of claim 1, wherein the compound is selected from the group consisting of semicarbazide, carbohydrazide, and sodium sulfite.

10. The method of claim 1, wherein the vicinal diol and a functional group of the compound which forms a bond with the aldehyde are in a molar ratio of at least 1:1.

11. A method for producing oxidation or reduction products of an aldehyde, comprising:
I) chemical or biocatalytic reaction of an aldehyde by reduction, oxidation, disproportionation or dehydration to give at least one reduction or oxidation intermediate, wherein the aldehyde is produced by the method of claim 1; and
II) optionally, further chemical or biocatalytic reaction of the at least one reduction or oxidation intermediate obtained in I) by reduction, oxidation, or addition.

12. The method of claim 1, wherein the vicinol diol in A) is glycerol.

13. The method of claim 1, wherein the vicinol diol in A) is 1,2-propanediol.

14. The method of claim 1, wherein the vicinol diol in A) is used in a concentration range from 0.2% by weight to 20% by weight based on the total reaction mixture.

15. The method of claim 1, wherein the vicinol diol and a functional group of the compound which forms a bond with the aldehyde are in a molar ratio of at least 1:1.2.

16. The method of claim 1, wherein the vicinol diol and a functional group of the compound which forms a bond with the aldehyde are in a molar ratio of at least 1:1.5.

17. The method of claim 11, wherein the at least one reduction or oxidation intermediate of I) is unsaturated.

18. The method of claim 11, wherein the reaction of I) is a chemical reaction.

19. The method of claim 11, wherein the reaction of I) is a biocatalytic reaction.

20. The method of claim 11, comprising:
II) further chemical or biocatalytic reaction of the at least one reduction or oxidation intermediate obtain in I) by reduction, oxidation, or addition.
